(19) United States
(12) Patent Application Publication
(10) Pub. No.: US 2026/0026780 A1
(43) Pub. Date: Jan. 29, 2026

(54) IMPROVED MEDICAL FOLLICLES ASSESSMENT DEVICE

(71) Applicant: PULSENMORE LTD., Omer (IL)

(72) Inventors: Lazar SONNENSCHEIN, Omer (IL); Ravit LEVI, Tel Aviv (IL); Minelu SONNENSCHEIN, Meitar (IL); Dotan MADAR, Ashkelon (IL)

(21) Appl. No.: 19/099,920

(22) PCT Filed: Jun. 7, 2023
(86) PCT No.: PCT/IL2023/050588

§ 371 Date: Jan. 30, 2025

(30) Foreign Application Priority Data
Aug. 25, 2022 (IL) 295946

(51) Int. Cl. A61B 8/12; A61B 8/00

(52) U.S. Cl. CPC A61B 8/12; A61B 8/4444

(57) ABSTRACT

A device adapted to monitor the follicles and to assess the thickness of the endometrium of a subject, comprises: a) an elongated member having a tip that houses an ultrasound probe; b) a handle adapted to be held in the hand of a subject; and c) a mechanism suitable to angularly displace said handle relative to said elongated member.

### FIELD OF THE INVENTION

The present invention relates to the field of medical devices. In particular, the invention relates to devices and a method for monitoring follicles and assessing the thickness of the endometrium.

### BACKGROUND OF THE INVENTION

In vitro fertilization (IVF) is a method of assisted fertilization in which an egg is combined with sperm outside the body (“in vitro”). IVF can be performed by collecting the contents from a woman's fallopian tubes or uterus after natural ovulation or ovarian stimulation. In most IVF procedures, the ovaries are stimulated to make the follicles grow and produce mature eggs. Ovarian follicles play a major part in every IVF cycle, and therefore monitoring the development of the ovarian follicles is a critical part of the IVF process. Nowadays, IVF patients undergo several pelvic (vaginal) ultrasound scans performed by specialized personnel, i.e., nurses or physicians, during the natural menstrual cycle or the ovarian stimulation phase to confirm that the dosage of medication given to them to promote ovulation is correct and to determine when they are ready for egg collection.

In addition, the thickness of the endometrium changes during a person's menstrual cycle, but other factors can prompt changes as well. It is therefore critical in many cases to measure the tissue thickness. The endometrium is the lining of the uterus. It is one of the few organs in the human body that changes in size every month throughout a person's fertile years. Each month, as part of the menstrual cycle, the body prepares the endometrium to host an embryo. Endometrial thickness increases and decreases during the process. Two hormones, estrogen and progesterone, prompt these cycles of endometrial growth, which is shed through menstruation if a pregnancy does not develop.

According to the Radiological Society of North America (RSNA), the endometrium is at its thinnest during menstruation, when it usually measures between 2-4 millimeters (mm) in thickness. The first half of the proliferative phase starts around day 6 to 14 of a person's cycle, or the time between the end of one menstrual cycle, when the bleeding stops, and before ovulation. At this phase, the endometrium begins to thicken and may measure between 5-7 mm. As the cycle progresses and moves towards ovulation, the endometrium grows thicker, up to about 11 mm. About 14 days into a person's cycle, hormones trigger the release of an egg. During this secretory phase, endometrial thickness is at its greatest and can reach 16 mm. Endometrial thickness is important in pregnancy. Healthcare experts link the best chances for a healthy, full-term pregnancy to an endometrium that is neither too thin nor too thick. This allows the embryo to implant successfully and receive the nutrition it needs. The endometrium gets thicker as the pregnancy progresses. Thus, monitoring and measuring the thickness is critical to a successful IVF and also for fertility preservation.

The monitoring includes assessing the number and size of the follicles on each ovary by ultrasound and by dedicated blood, saliva, or urine tests that measure the concentrations of relevant hormones. When the follicles are ready and are of the right size, around 18-20 mm, a trigger of hCG hormone injection is administrated. This trigger stimulates the follicles to discharge the mature eggs. Specialists then collect the mature eggs at a medical facility.

Because timing is essential in all IVF procedures, there is a need for daily monitoring of the ovarian follicles and the thickness of the endometrium, which creates a substantial burden on the patients and on the medical personnel and equipment. It is therefore clear that it would be highly desirable to be able to obviate the need for frequent visits to the medical facility where the assessment of follicles development is performed, thus reducing the burden and costs on the patient and the system alike.

In pursuit of the abovementioned aim, a device was developed which substantially reduces the need to perform follicle monitoring and its assessment, as well as the thickness of the endometrium at a medical facility by specialized personnel, which is suitable to be used for assessment purposes for IVF and Embryo Transfer (IVF-ET), as well as other fertility-related procedures. These may include, for instance, monitoring of spontaneous ovulation, natural preservative procedures by determining when there is no danger of pregnancy, and any other medical process requiring such monitoring.

The abovementioned device is the subject of co-pending Israeli patent application nos. 285798 filed Aug. 23, 2021, and 293114 filed May 12, 2022, by the same applicant hereof, the whole description of which is incorporated herein by reference. A device according to one embodiment of the abovementioned patent applications is shown in FIG. 1. In this embodiment, grip 112 is provided with actuation switch 112, which operates the ultrasound probe located in tip 113. Since handle or grip 111 is held in the patient's hand during the operation of the ultrasound device, a simple application of pressure or release thereof turns the probe on or off, as the case may be. However, in embodiments of the invention there is no need to keep applying pressure on switch 112 for the ultrasound probe to be active. An additional or alternative pressure switch can be located at the backside of the grip (not shown in FIG. 1). Suitable switches and switch assemblies adapted for this purpose are well known in the art and therefore are not discussed herein for the sake of brevity.

The structure of the device of the invention can be, but not necessarily is, monolithic. For instance, in some embodiments, the device can be made of separate parts adapted to be assembled, some of which may be detachable. For instance, numerals 114, 115, and 116 may each represent an assembly/disassembly line, where the device can be taken apart for maintenance or parts replacement.

The device is adapted to identify follicle proprieties and the thickness of the endometrium. The device is connected, wirelessly or via a wired connection, to a smartphone or the like smart device and allows a layman operator to produce ultrasound images without the need for a specialized ultrasound operator to be present in person, e.g., in the convenience of their home. The device may connect to portable devices such as smartphones, tablets, or other suitable devices via wireless or wired connections. Wireless connections may include, for example, Bluetooth or Wi-Fi, WIFI 5/6/7/8, UWB (ultra-wideband), or the like. In one embodiment of the invention, the follicles identification and the determination of the thickness of the endometrium are performed automatically using image processing and related processes. Alternatively, these determinations are carried out remotely by a specialist who inspects the images generated by the device. Images are transferred to the smart device coupled with the device of the invention via wire or wirelessly and then transmitted to the specialist for evaluation.

The device enables obtaining valid clinical data by instructing the patient to move the device in simple hand movements. Accordingly, without the need for any technical background, patients are able to scan and automatically send the images to the physician or other technician.

As will be understood by the skilled person, the device of the invention has a defined grip, which also defines the position of the ultrasound probe. For instance, when holding handle 111 of the device of FIG. 1, the probe's rotation is limited by the movement of the user's hand and, therefore, the direction in which the probe is scanning is essentially defined, as opposed to prior art devices in which the elongated device may rotate freely and as such just by looking at the images acquired it is not possible to know whether scanning took place along a vertical, horizontal, or intermediate line.

As used herein, the term “monitoring” refers to both visual inspection and parameter measurements, as the case may be. For instance, monitoring the development of follicles will, in many cases, involve measuring their size, as when IVF procedures are involved, but in some cases, it may be sufficient to ascertain qualitatively that follicles are developing, as may be, for example, in some cases when natural ovulation is to be confirmed, even though the measurement of follicles size may be desirable or necessary also when monitory natural ovulation. The same applies to the monitoring of the endometrium.

Therefore, providing means by which the user can operate the monitoring device safely and conveniently is also an important factor in the success of the monitoring. The inventors have found that an improved user experience can be provided by allowing the user to self-control the depth of introduction of the device, such that it is not insufficient, which may adversely affect the quality of the recorded images, and not too deep, which may be unsafe.

Furthermore, the inventors have also found that allowing a user to choose the angle at which the device is held in the user's hand relative to the axis of penetration of the device may also contribute to the user's experience and the quality of the monitoring.

It is therefore a purpose of the present application to provide a device that achieves the aforesaid goals in a convenient and easy-to-operate manner.

It is a further purpose of the invention to provide a device and a method that reduce the load currently imposed on medical sites, thereby saving time and money for both patients and the health care system.

It is yet another object of the invention to provide a device that can be useful and easy to use for monitoring during various stages of the menstrual cycle, as well as an aid in other fertility procedures.

### SUMMARY OF THE INVENTION

The invention relates to a device adapted to monitor the follicles and to assess the thickness of the endometrium of a subject, comprising:
a) an elongated member having a tip that houses an ultrasound probe;
b) a handle adapted to be held in the hand of a subject; and
c) a mechanism suitable to angularly displace said handle relative to said elongated member.

In embodiments of the invention the angular displacement mechanism is housed in a shell positioned between the elongated member and the handle. In some embodiments, the device is provided with an indexing arrangement adapted to indicate a current angular displacement.

The device may comprise a mechanism that is adapted to perform stepwise angular displacements, or a mechanism that is adapted to perform continuous angular displacements. In embodiments of the invention, the angle between the central axis of the elongated member and that of the handle can be changed between 0° and 180°. Furthermore, the angular displacement mechanism may comprise gear components.

The invention is also directed to a device adapted to monitor the follicles and to assess the thickness of the endometrium of a subject, comprising:
a) an elongated member having a tip that houses an ultrasound probe;
b) a handle adapted to be held in the hand of a subject; and
c) a limiting element adapted to prevent a user from inserting the elongated member at a depth greater than appropriate.

The limiting element may comprise a ringlike member that can slide along the length of the elongated member, and in some embodiments an element is provided, which is adapted to prevent the involuntary sliding of the ring. The involuntary sliding of the ring can be prevented, for instance, by creating friction between the interior of the ring and the surface of the elongated member, e.g., by providing a rough surface formed along a length of the elongated member.

In some embodiments it is advantageous to provide one or more rulers on the elongated member, said rulers comprising indicia indicative of a length.

The depth-limiting element can be of many different kinds and, in one embodiment it comprises a telescopic section of the elongated member.

Some embodiments also comprise a stabilizing element adapted to keep a protective cover from accidentally sliding along the elongated member.

### BRIEF DESCRIPTION OF THE DRAWINGS

In the drawings:

FIG. 1 is a perspective view of an illustrative device according to IL285798 or IL293114;

FIG. 2 is a perspective view of a device according to one embodiment of the invention;

FIG. 3 is a perspective view of the device of FIG. 2 in an alternative position;

FIG. 4 is a perspective view of the device of FIG. 2 in yet another alternative position;

FIG. 5 is an exploded view of the device of FIG. 2;

FIG. 5A shows the view of FIG. 5, seen from behind;

FIG. 6 is a side view of a device according to another embodiment of the invention;

FIG. 7 is a side view of a device according to yet another embodiment of the invention;

FIG. 8 is a top view of a device according to yet another embodiment of the invention;

FIG. 9 is a bottom view of a device according to a further embodiment of the invention;

FIG. 10 is a top view of a cross-section of the device of FIG. 9;

FIG. 11 is a perspective view of yet another embodiment of the invention; and

FIG. 12 (A and B) show another embodiment of the invention with a telescopic depth-limiting mechanism.

### DETAILED DESCRIPTION OF THE INVENTION

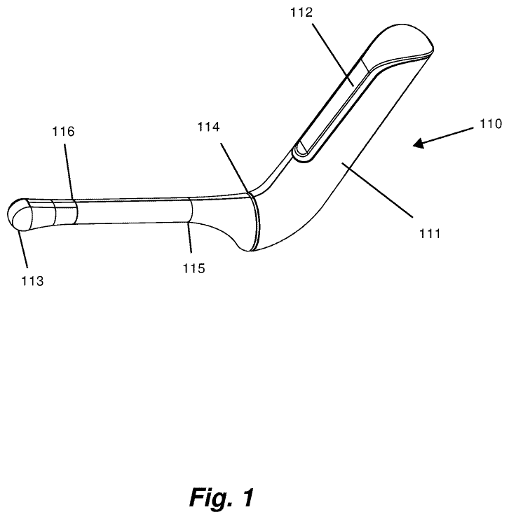

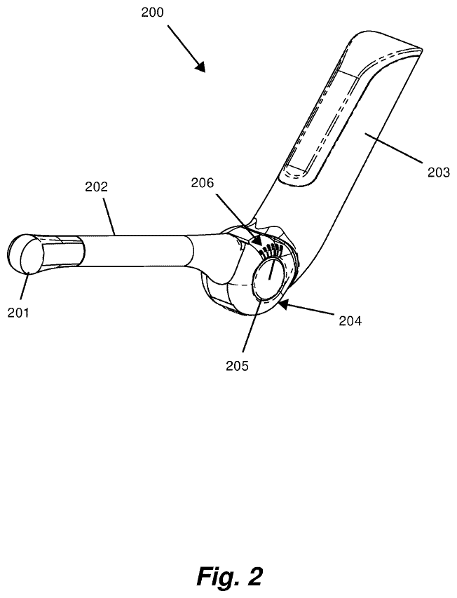

The device is adapted to identify follicle proprieties and the thickness of the endometrium and possesses all the elements and components referred to above with reference to IL285798 and IL293114 and in the description thereof. FIG. 2 shows a device 200 according to one embodiment of the invention, with an ultrasound probe housed in tip 201 located at the distal end of the device and of the elongated portion 202 thereof, which is to be inserted in the vagina for ultrasound scanning. Like in the device of FIG. 1, a handle or grip 203 is the portion that the user holds during scanning. Handle 203 may hold various components of the system, such as communication elements, image processing, and power supply, which are not discussed herein in detail for the sake of brevity since they do not influence the mechanical elements of the invention. Similarly, elongated member 202 may contain wiring or wireless components to transmit data generated by the ultrasound probe to the handle or to an external device.

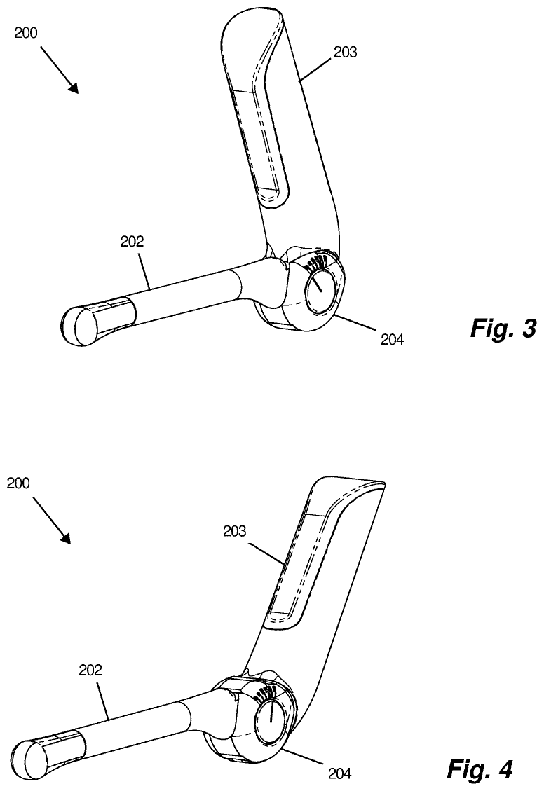

Unlike the device of FIG. 1, in the device of FIG. 2, handle 203 is not fixed relative to elongated portion 202 but rather is capable of acquiring different positions by means of a mechanism (not shown) contained in housing 204, a detailed example of which will be discussed hereinafter. The bottom portion of handle 203 can rotate around the axis of plug 205, and the rotation may be performed stepwise or continuously to reach different positions indicated by dial 206. FIG. 3 shows the device of FIG. 2 with the handle positioned at a smaller angle than that of FIG. 2, relative to elongated member 202, and FIG. 4 shows the same device with the handle positioned at a larger angle than that of FIG. 2, relative to elongated member 202.

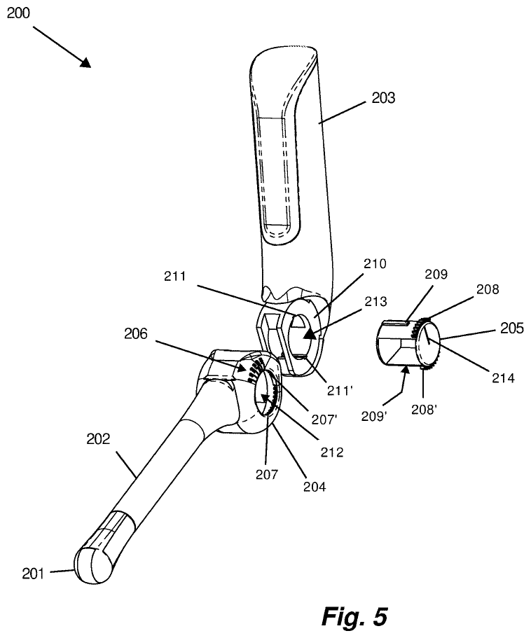

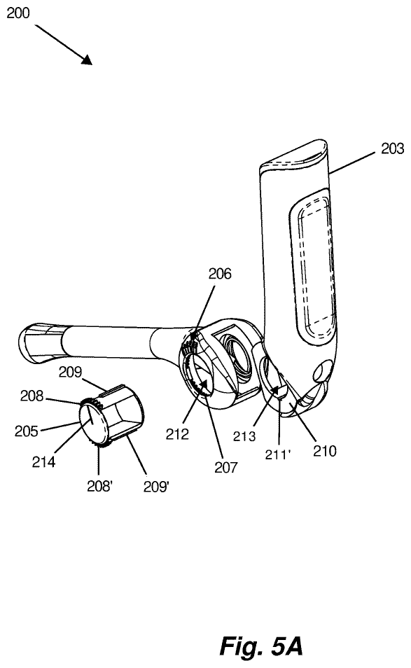

Looking now at FIG. 5., The device of FIG. 2 is shown in an exploded view, which illustrates a rotation mechanism according to one embodiment of the invention. As will be appreciated by the skilled person, many different mechanisms can be devised, which will allow performing the angular movement of handle 203 relative to elongated member 202, and the mechanism shown in FIG. 5 is just one of many such alternatives.

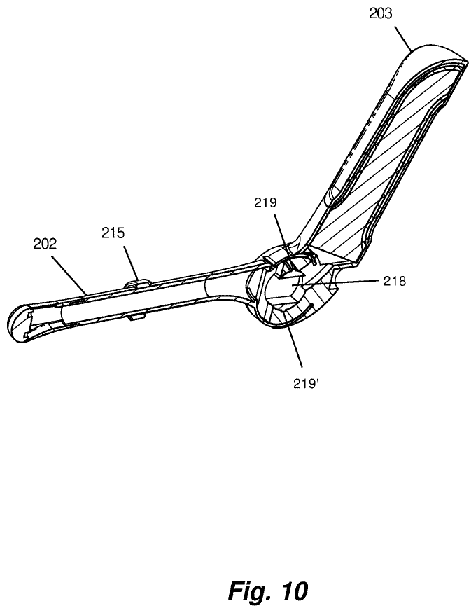

In this embodiment, housing 204 is provided with toothed portions 207 and 207′, which in the assembled position interact with tooths 208 and 208′ of plug 205. Of course, as said above, many other gearing alternatives can be devised by the skilled person. Plug 205 has elongated ridges 209 and 209′ on the bottom, which is not seen in the figure and is indicated by an arrow pointing at its location. In turn, the bottom portion 210 of handle 203 is sized to fit into the hollow portion of housing 204 and is provided with slots 211 and 211′, into which ridges 209 and 209′ respectively fit. Thus, in the assembled position, when plug 205 passes through openings 212 and 213 and ridges 209 and 209′ engage slots 211 and 211′, teeth 208 and 208′ engage teeth 207 and 207′, and the device is ready to be rigidly rotated around the axis of plug 205, by the limited movement of teeth 208 and 208′ against teeth 207 and 207′. Dial 206 may, in one embodiment, indicate the angle between handle 203 and elongated portion 202 at the relevant location by positioning line 214 provided on plug 205 against the appropriate location on the dial. The values on the dial can of course be of any other kind, for instance, simple running numbers or color codes, since their purpose is to allow the user to determine the position that is most comfortable to her for operating the device. Opposite plug 205, a cap 218 can be provided (not shown in FIG. 5 but shown in FIG. 10), which engages slots 211 and 211′ from the opposite side, with ridges 219 and 219′.

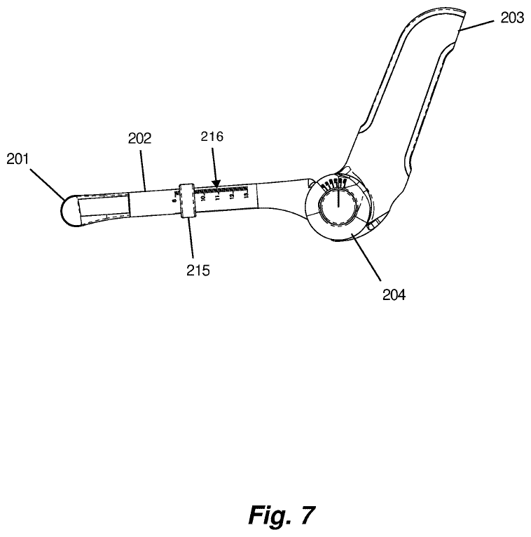

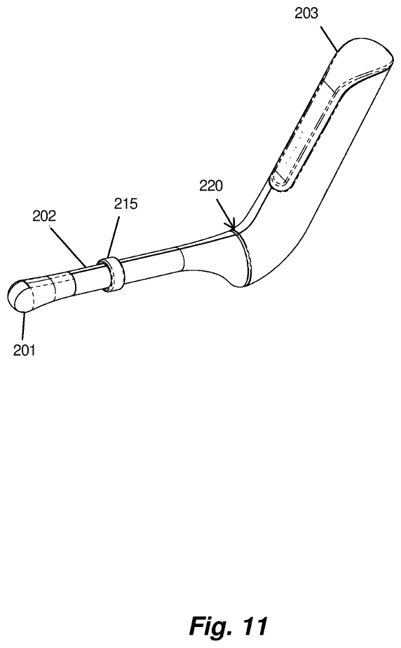

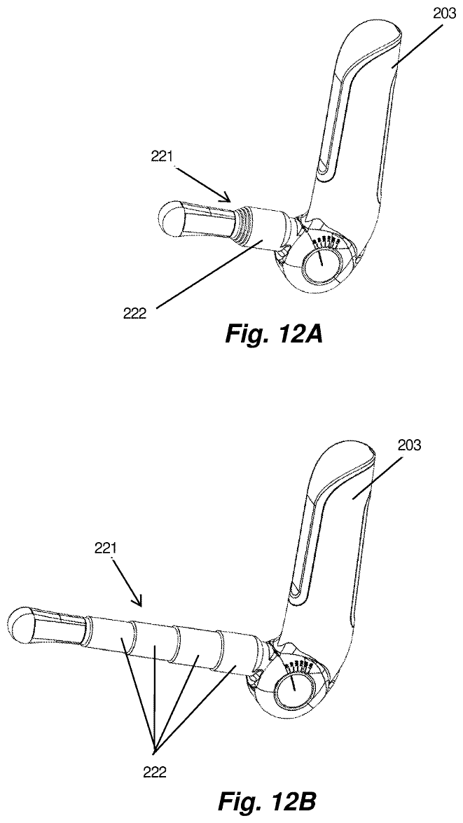

FIG. 11 is a further embodiment of the invention, which is provided with a depth-limiting mechanism 215, which may be coupled to any of the slides and/or the rough surfaces discussed with reference, for instance, to FIGS. 7-9. This embodiment does not comprise the rotation assembly of the previous figures, and the position of handle 203 relative to elongated member 202 is fixed. For the sake of simplicity, the depth-limiting mechanism will be referred to as a limiting “ring,” it being understood that the ring is merely one possible embodiment of said depth-limiting mechanism and that alternative mechanisms can be used, such as, for instance, the one described hereinafter with reference to FIG. 12.

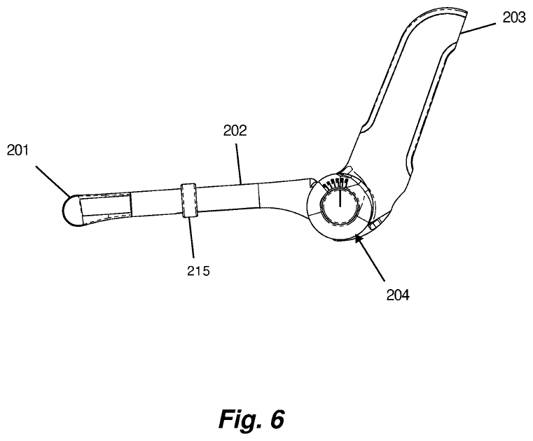

FIG. 6 shows a device according to another embodiment of the invention. This embodiment ensures that the user inserts the device at an appropriate depth into the vagina since inserting it too much may cause damage and pain, and inserting it too little may result in low-quality or useless images. This result is achieved in this embodiment by a limiting ring 215, adapted to slide along the length of elongated member 202 and remain at the chosen location by friction or any other suitable means. As will be easily understood by the man of the art, many alternative ways exist to position the ring (or another depth-limiting elements), such as fastening means, such as screws, bolts, and nuts, which are provided in the ring apply a pressure on the surface of elongated member 202. More advanced ways of determining how to limit the depth of penetration may include sensors, such as an EDI current sensor, with a memory that stores the desired depth and, in further uses, shows on a screen (e.g., of a smartphone) the location and the distance remaining to the desired depth. Alternative sensors can of course be employed, such as magnetic sensors, photo diode sensor.

An alternative way to prevent an excessive depth of penetration is provided according to other embodiments of the invention. In these embodiments, an ultrasound sensor is used to provide an alert when the tip of the device is close to touching the mucosa, or when a predetermined threshold of pressure against the tissue is exceeded. For this purpose, embodiments of the invention make use of elements of the ultrasound transducer. The skilled person will easily device software adapted to operate the abovementioned sensors as well as interfaces for the patient and the physician or technician, which facilitate the insertion of the device to the correct depth.

On the first use, the patient determines the appropriate location of the limiting ring 215, which determines the length of insertion, either by herself, following instructions, or with the help of a health professional. In the following uses, ring 215 will determine the insertion depth and ensure that it is constant and appropriate based on the initial determination. In some embodiments two rings 215 (the second ring not shown) are present to allow for different insertion depth in case of asymmetry in the organs.

FIG. 7 shows another embodiment of the invention, which employs a limiting ring 215 together with a ruler 216. In this embodiment, the device allows the user to obtain a reading representative of the correct position of ring 215 without the need to fix the ring's location. Accordingly, once the correct location of ring 215 on the slide is determined, it is possible to reposition it at the same position before each use and still use ring 215 to indicate the desired penetration depth. Ruler 216 may be simply printed or can be embossed on elongated member 202. The embossing, when present, can also be used to apply friction to the portion of ring 215 that comes into contact with it, particularly if said surface is rough. Of course, in some embodiments of the invention ring 215 can be omitted and the patient may determine the depth of insertion simply by looking at the ruler.

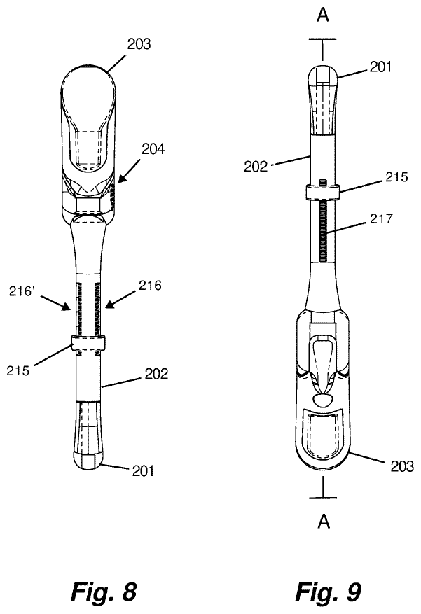

FIG. 8 shows an alternative embodiment to that of FIG. 7, in which two rulers, 216 and 216′, are provided, each on one side of the elongated member 202, to facilitate reading by the user.

FIG. 9 shows a further embodiment of the invention, in which a rough surface 217 is provided at the bottom of the elongated member 202, its roughness being sufficient to prevent the ring 215 from being displaced without the application of some force. This, again, can be achieved by providing a rough surface also in the inside portion of ring 215, by providing a protruding ridge therein, or by any other suitable method known to the man of the art.

FIG. 10 is a cross-section of the device of FIG. 9, taken along the AA line, which shows ring 215 in cross-section. It also shows cap 218, referred to with reference to FIG. 5 above, which is not shown in the previous figures.

FIG. 11 shows a further embodiment of the invention, in which the mechanism housing 204 of FIG. 2 does not exist, and the handle is rigidly fixed relative to elongated member 202. In this embodiment, a ring 202 is provided, as in, for example, FIG. 7. Alternative embodiments may have one or more rulers, such as 216 of FIG. 7, and/or a rough surface for the controlled displacement of ring 2015, such as, for instance, surface 217 of FIG. 9.

An additional feature of embodiments of the invention is the provision of a stabilizing element such as, for instance, the protrusion indicated by numeral 220 in FIG. 11, which may be located at a disassembly line, as in 114 of FIG. 1, or elsewhere along elongated element 202. The purpose of this protrusion is to help keep in place a protective sheath (not shown) that can be placed over the elongated member 202 to avoid infection and reduce the need for cleaning, and high level of disinfection or sterilization. Using a protective cover for medical devices to be inserted into a lumen is common practice in the medical field and providing a stabilizing element as provided by this embodiment reduces the risk of the protective cover sliding away. Of course, the stabilizing element does not have to have a circular shape and can be of any other shape suitable to apply a holding force on a protective cover to hinder its sliding away.

FIG. 12 shows a device according to one embodiment of the invention in which the depth of penetration of the probe is determined by the movement of sections 222 of a telescopic elongated member 221, in which e few elements are shown for the purpose of illustration. FIG. 12A shows the device in a short (folded) position, and FIG. 12B shows it is a fully expanded position. By choosing the number of sections to be displaced away from the handle, the length of penetration is limited.

As will be appreciated by the skilled person, the embodiments described above are merely illustrative, and many modifications can be provided thereto. For instance, instead of a rough surface 217, a stepwise positioning element can be provided. Moreover, the rotation mechanism can be of many different types, allowing continuous movement (e.g., using friction-based elements) or stepwise movement, either purely mechanical or by an electric motor. The alternatives by which the purposes of the invention may be achieved are numerous, and the skilled person will easily devise viable alternatives to the ones described above for the purpose of illustration.

1. A device adapted to monitor the follicles and to assess the thickness of the endometrium of a subject, comprising:
a) an elongated member having a tip that houses an ultrasound probe;
b) a handle adapted to be held in the hand of a subject; and
c) a mechanism suitable to angularly displace said handle relative to said elongated member;
wherein the angular displacement mechanism is housed in a housing positioned between the elongated member and the handle; and
wherein the device is provided with an indexing arrangement adapted to indicate a current angular displacement.

2. The device according to claim 1, wherein the mechanism is adapted to perform stepwise angular displacements.

3. The device according to claim 1, wherein the mechanism is adapted to perform continuous angular displacements.

4. The device according to claim 1, wherein the angle between the central axis of the elongated member and that of the handle can be changed between 0° and 180°.

5. The device according to claim 1, wherein the angular displacement mechanism comprises gear components.

6. The device of claim 1, further comprising a limiting element adapted to prevent a user from inserting the elongated member at a depth greater than appropriate.

7. The device according to claim 6, wherein the limiting element comprises a ringlike member that can slide along the length of the elongated member.

8. The device according to claim 7, comprising an element adapted to prevent the involuntary sliding of the ring.

9. The device of claim 8, wherein the involuntary sliding of the ring is prevented by creating friction between the interior of the ring and the surface of the elongated member.

10. The device of claims 9, comprising a rough surface formed along a length of the elongated member.

11. The device according to claim 6, comprising one or more rulers provided on the elongated member, said rulers comprising indicia indicative of a length.

12. The device according to claim 6, wherein the limiting element comprises a telescopic section of the elongated member.

13. The device according to claim 1, comprising a stabilizing element adapted to keep a protective cover from accidentally sliding along the elongated member.
